(19) United States
(12) Patent Application Publication
(10) Pub. No.: US 2011/0272282 A1
(43) Pub. Date: Nov. 10, 2011

(54) SYSTEMS AND METHODS FOR ENHANCED SCODA

(75) Inventors: Andrea Marziali, North Vancouver (CA); David John Broemeling, Vancouver (CA); Joel Pel, Vancouver (CA); Jason Donald Thompson, Vancouver (CA); Jaryn Perkins, Vancouver (CA); Thomas Willis, San Francisco, CA (US); Herbert Heyneker, San Francisco, CA (US); Darren Gray, La Jolla, CA (US); Carolina Tropini, Redwood City, CA (US)

(73) Assignee: University of British Columbia, Vancouver, British Columbia (CA)

(21) Appl. No.: 13/128,387

(22) PCT Filed: Nov. 10, 2009
(86) PCT No.: PCT/CA09/01648

§ 371 Date: Jul. 20, 2011

Related U.S. Application Data
(60) Provisional application No. 61/193,250, filed on Nov. 10, 2008.
(60) Provisional application No. 61/193,975, filed on Jan. 14, 2009.

(51) Int. Cl. C07K 1/26; C25B 9/00

(52) U.S. Cl. 204/456; 204/450; 204/600; 204/547

(57) ABSTRACT

Methods and apparatus for concentrating particles may be applied, for example, to concentrating DNA, RNA, proteins and the like. Proteins may be pre-treated to facilitate concentration by scodaphoresis. The pre-treatment may comprise, for example, heating or chemical treatment to denature and/or apply a net charge to the protein, binding handle particles to the protein and combinations thereof. High-conductivity samples may be subjected to a conductivity-reduction step to facilitate electrical injection of target particles into scodaphoresis media. The conductivity-reduction step may comprise a buffer exchange process or a salt extraction process, for example. Methods and apparatus can allow two or more different types of target particles to be extracted from the same sample and separately concentrated. These various aspects may be applied individually or in any combination.

### REFERENCE TO RELATED APPLICATION

For the purpose of the United States of America, this application claims the benefit under 35 U.S.C. §119(e) of U.S. Patent Application No. 61/193,250 filed 10 Nov. 2008; and U.S. Patent Application No. 61/193,975 filed 14 Jan. 2009, both of which are hereby incorporated herein by reference in their entireties.

### TECHNICAL FIELD

The invention relates to the induced movement of particles such as proteins and other molecules through media such as gels and other matrices. Some embodiments provide methods and apparatus for selectively concentrating particles of interest. Some embodiments relate to scodaphoresis methods and apparatus.

### BACKGROUND

Scodaphoresis (or “SCODA”) is an approach that may be applied for concentrating and/or separating particles. SCODA may be applied, for example, to DNA, RNA and other molecules. The following background discussion of SCODA is intended to provide examples that illustrate principles of SCODA and is not intended to impose any limitations on the constitution, makeup or applicability of SCODA methods and apparatus generally.

SCODA is described in:
(1) US Patent Publication No. 2009/0139867 entitled “Scodaphoresis and methods and apparatus for moving and concentrating particles”;
(2) PCT Publication No. 2006/081691 entitled “Apparatus and methods for concentrating and separating particles such as molecules”;
(3) PCT Publication No. WO 2009/094772 entitled “Methods and apparatus for particle introduction and recovery”;
(4) Marziali, A.; Pel, J.; Bizotto, D.; Whitehead, L. A., “Novel electrophoresis mechanism based on synchronous alternating drag perturbation”, Electrophoresis 2005, 26, 82-89;
(5) Broemeling, D.; Pel, J.; Gunn, D.; Mai, L.; Thompson, J.; Poon, H.; Marziali, A., “An Instrument for Automated Purification of Nucleic Acids from Contaminated Forensic Samples”, JALA 2008, 13, 40-48; and
(6) Pel, J.; Broemeling, D.; Mai, L.; Poon, H.; Tropini, G.; Warren, R.; Holt, R.; Marziali, A., “Nonlinear electrophoretic response yields a unique parameter for separation of biomolecules”, PNAS 2008, vol. 106, no. 35, 14796-14801,
   all of which are hereby incorporated herein by reference.

SCODA can involve providing a time-varying driving field that applies forces to particles in some medium and a time-varying mobility-altering field that affects the mobility of the particles in the medium. The mobility-altering field is correlated with the driving field so as to provide a time-averaged net motion of the particles. SCODA may be applied to cause selected particles to move toward a focus area.

Some modes of SCODA exploit the fact that certain particles in appropriate media exhibit non-linear responses to electric fields. In such modes, suitably time-varying electric fields can cause certain types of particles to be focused or concentrated at locations within the medium. In many practical cases, the instantaneous velocity of a particle in a medium under the influence of an electric field is given by:

\vec{v}=μ\vec{E} (1)

where \vec{v} is the velocity of the particle, \vec{E} is the electric field and μ is the mobility of the particle in the medium given, at least approximately, by:

μ=μ₀+κ|\vec{E}| (2)

where μ₀ and κ are constants. Particles with larger values for κ tend to be focused more strongly than particles with smaller values for κ.

In some cases, SCODA is performed by providing an electrical field having a rotating component and a quadrupole perturbation. The rotating component may be specified, for example, by:

Eₓ=E cos(ωτ) (3)

and

E_{y}=E sin(ωτ) (4)

where E is a magnitude of the electric field component that rotates at an angular frequency ω, and Eₓ and E_{y} are respectively x- and y-components of the rotating electrical field. The perturbing quadrupole component may be specified, for example, by:

dE_{χ}=−dEₓx cos(2ωτ) (5)

and

dE_{γ}=dE_{q}y cos(2ωτ) (6)

where dEₓ and dE_{y} are respectively x- and y-components of the perturbing electrical field, x and y are distances from an origin at the center of the quadrupole field pattern and dE_{q} is the intensity coefficient of the perturbing quadrupole field. With this SCODA field, the average radial velocity of a particle toward a focus location can be shown to be given by:

$$\overline{\vec{v}} = \frac{κ {EdE}_{q}}{4} \vec{r} \qquad ( 7 )$$

where \vec{r} is a vector pointing toward the focus location and having a magnitude equal to the distance of the particle from the focus location.

The size of a spot into which particles can be focused depends upon κ as well as on the rate at which the particles can diffuse in the medium as follows:

$$\frac{1}{R} ∝ \sqrt{\frac{κ}{D}} \qquad ( 8 )$$

where R is a radius of the focused spot and D is a diffusion coefficient.

Molecules having large values of \sqrt{κ/D} may focus in the medium under SCODA conditions, and are selectively concentrated within smaller radius distances R relative to molecules with smaller values of \sqrt{κ/D}.

A limitation of SCODA applications in which electric fields are applied to inject target molecules from a sample into a gel or other SCODA medium is that the applied electric fields can cause electrical currents. The more electrically conductive the sample, the larger are the electric currents generated for a given electrical field strength within the sample. The electric currents result in heating which can damage target molecules and/or the SCODA medium. Excessive heating can also impair the efficacy of SCODA.

### SUMMARY

This invention has a number of aspects that may be applied independently or in combination with one another.

In addition to the exemplary aspects and embodiments described above, further aspects and embodiments will become apparent by reference to the drawings and by study of the following detailed description.

### BRIEF DESCRIPTION OF THE DRAWINGS

Example embodiments are illustrated in the accompanying drawings. The illustrated embodiments are intended to be illustrative and not restrictive.

FIG. 1A is an image of an experimental setup for SCODA concentration of proteins according to one illustrative embodiment.

FIG. 1B is an image of an experimental result of SCODA concentration of proteins according to another illustrative embodiment.

FIG. 2A is a table of example electrode voltage values for quadrupole injection and SCODA concentration of negatively charged particles according to another illustrative embodiment.

FIG. 2B is a table of example electrode voltage values for SCODA concentration of positively charged particles according to another illustrative embodiment.

FIG. 3 is an image of the molecules concentrated in the SCODA experiment run on an SDS-PAGE gel according to another illustrative embodiment.

FIG. 4 is a block diagram illustrating an apparatus that may be applied for SCODA concentration of a plurality of different particles from a single sample according to another illustrative embodiment.

FIG. 5 is a block diagram illustrating a SCODA controller according to another illustrative embodiment.

FIG. 6 is a flow diagram illustrating an example method for concentrating a sample from within a high conductivity mixture according to another illustrative embodiment.

FIG. 7 is an image of a DC electrophoresis gel showing DNA recovered by SCODA from a buffer sample that underwent a buffer exchange process according to another illustrative embodiment.

FIG. 8 is a flow diagram illustrating a method for concentrating a sample from within a high conductivity mixture according to another illustrative embodiment.

FIG. 9 is an image of a DC electrophoresis gel showing DNA recovered by SCODA from lysed E. coli using a buffer exchange process according to another illustrative embodiment.

In the drawings, identical reference numbers identify similar elements or acts. The sizes and relative positions of elements in the drawings are not necessarily to scale. For example, the shapes of various elements and angles are not drawn to scale, and some of these elements are arbitrarily enlarged and positioned to improve drawing legibility.

### DESCRIPTION

In the following description:
Known elements may not be shown or described in detail to avoid obscuring the disclosure.
Specific details are provided to facilitate thorough understanding of various disclosed example embodiments. However, embodiments may be practiced without one or more of these specific details, or in other combinations with other methods, components, materials, etc.
References to “one embodiment” or “an embodiment” mean that a particular feature, structure or characteristic described in connection with the embodiment is included in at least one embodiment.
Phrases like “in one embodiment” or “in an embodiment” do not all refer to the same embodiment. Furthermore, the particular features, structures, or characteristics of the various embodiments described herein may be combined in any suitable manners to yield additional embodiments.
The headings and Abstract of the Disclosure are for convenience only and do not interpret the scope or meaning of the embodiments or any terms used herein.

Unless the context clearly requires otherwise, throughout the specification and claims which follow:
the word “comprise” and variations thereof, such as, “comprises” and “comprising” are to be construed in an open, inclusive sense, that is as “including, but not limited to.”
the singular forms “a,” “an,” and “the” include plural referents.
the term “or” is employed in its inclusive sense “and/or”.

### Enhanced SCODA Focusing

As noted above, where the mobility μ of a type of particle is given, at least approximately, by Equation (2) particles of types having larger values for κ tend to be focused more strongly than are particles of types having smaller values for κ. κ may be described as a ‘non-linearity coefficient’ or a ‘coefficient of field dependence of the particle's mobility’. One aspect of the invention provides SCODA methods and devices in which the value of κ for target particles is increased. In some embodiments, the target particles are biomolecules. In some specific embodiments, the target particles comprise one or more proteins. In some embodiments, the SCODA driving and mobility-altering fields comprise electrical fields.

This aspect provides one or more process steps that alter κ for target particles. The process steps comprise one or more of:
physical treatment which increases κ for target particles and/or decreases κ for non-target particles;
chemical treatments which increases κ for target particles and/or decreases κ for non-target particles; and
affixation of molecules or other particles to target particles and/or non-target particles that has the effect of increasing κ for target particles and/or decreasing κ for non-target particles.

Such process steps can alter physical properties of particles (which may be molecules, for example). The altered properties that contribute to the alteration of κ may include one or more of (but are not limited to): electric charge, shape, degree of folding, drag, and conformation.

One example of a physical process step that can increase κ for a target particle is heat treatment. The heat treatment may include, for example, heating a sample to a temperature and for a period of time sufficient to cause a change in target particles in the sample. In some embodiments the sample is brought to a boil or is heated by thermal contact with a boiling water bath. Heating can be particularly effective for altering κ where the target particle is a protein or other molecule that becomes denatured and/or experiences a change in the degree of folding as a result of the heating.

Examples of a chemical process step that can increase κ for a target particle are treatment with chemicals that are effective to impart a net electric charge to target particles and/or alter a configuration of the target particles. In some embodiments the target particles are molecules and the chemical treatment denatures and/or changes the degree of folding of the target particle molecules.

The chemical treatment may include, for example, treatment with one or more of: tris-glycine, dithiothreitol, and sodium sodecyl sulfate. In some embodiments the target particles comprise disulfide bonds and the chemical treatment comprises treatment with a chemical that breaks disulfide bonds. In some embodiments the chemical treatment comprises treatment with a detergent such as a suitable anionic surfactant.

Molecules or other particles may be affixed to target particles in various ways. For example, “handle” molecules, having a specific response to SCODA fields, may be attached to “target” molecules by one or more of:
a linking agent which may comprise, for example, a biomolecule such as an antibody, biotin-avidin complex, an RNA aptamer,
bonding between the handle and target particles, the bonding may, for example, comprise hydrogen bonding, ionic bonding, or covalent bonding,
hydrophobic interactions between the handle and target particles.
other chemical or physical connections.

Target particles to which handle molecules may be attached may comprise, but are not limited to, biomolecules such as proteins, enzymes and nucleic acids such as RNA and DNA. In some example embodiments the handle molecules comprise nucleic acids or proteins (the proteins may be modified so as to be readily focused by a SCODA field). In some embodiments the handle molecules comprise a marker such as a dye or the like.

In some embodiments the handle particles or a linking agent provided to link handle particles to target particles have a specific affinity for particular target particles. For example:
Where the target particles comprise a particular protein, the handle particles may comprise an antibody that interacts specifically with the target particles. The handle particles may comprise, for example, the antibody chemically bonded to a nucleic acid.
Where the target particles comprise a particular DNA or RNA sequence the handle particles may comprise a DNA or RNA sequence that is complementary to the sequence of the target particles.
   In such embodiments, a specific protein or other target particle may be moved or concentrated by SCODA fields acting on the handle particles while other particles similar to the target particles which do not bind to the handle particles (or do not bind as strongly to the handle particles) are not concentrated (or not concentrated very much) by the SCODA fields.

Where the handle particles have an affinity for target particles, the handle particles may be attached to the target particles by mixing handle particles into a sample containing the target particles. For example, where the target particles comprise a particular protein, the handle particles may comprise a strand of nucleic acid (e.g. DNA or RNA) linked to an antibody that binds to the protein. The antibody-linked nucleic acid can be mixed with a sample containing the protein targeted by the antibody. The resulting sample can then be processed with SCODA to concentrate the targeted protein at a point in a medium. Such Focusing may occur even in cases where the protein itself is electrically neutral or, for some other reason, is not focused very much or at all by the applied SCODA fields.

The foregoing techniques may be applied to improve the selectivity of SCODA Focusing for selected target particles and/or to improve the degree to which SCODA focuses target particles. In some embodiments, two or more of the above techniques are applied. For example, in one embodiment a sample is prepared for SCODA by a physical or chemical treatment step which alters target particles followed by a process step which selectively attaches handle particles to the altered target particles. The altered target particles are then concentrated by SCODA.

Under suitable preparation/lysis conditions, SCODA may be applied to concentrate target particles such as biomolecules (e.g. molecules of nucleic acid, proteins, enzymes and the like) from a wide range of samples. The samples may include, for example, human or animal samples including: blood, tissue, urine, stool, hair, biopsy, sputum, lavage fluids, discharge, mucus, skin; environmental samples such as: food, water, soil, collected aerosols, plant samples; archeological samples such as: bone, fossil, tar sands, tar pit, ice cores; and so on.

In some embodiments, target particles are given a selected value for the parameter κ/D by one or some combination of physical treatment steps, chemical treatment steps and affixation of handle particles and then the target particles are separated from other particles on the basis of differences in the parameter κ/D.

### SCODA Concentration of Proteins

The above techniques are useful for enhancing SCODA focusing of proteins. Protein molecules, including fragments of proteins, tend to have relatively low net electric charge and are typically folded in a way that limits the amount of conformational change that results from changes in the strength of applied electric fields. Consequently, proteins tend to have low values for x and tend not to focus very well under electrophoretic SCODA.

Separation and/or concentration of a protein in electrophoretic SCODA may be facilitated by subjecting the protein to one or more physical and/or chemical treatments that increase the value of κ. In some embodiments these treatments denature the proteins and cause the net electric charge on molecules of the protein to increase. Samples containing proteins may comprise, for example, total protein from a cell, or group of cells such as a cell culture. In some embodiments samples containing proteins are prepared according to a protocol for preparing samples for sodium dodecyl sulphate-polyacrylamide gel electrophoresis (SDS-PAGE).

The SCODA separation and/or concentration of the protein may also be improved by coupling the protein to another molecule (a ‘handle’), such as DNA or RNA, that has a significantly greater value of κ than the protein. In some cases the aggregate particle (protein attached to handle molecule) also has a smaller value for D than the protein alone. By increasing κ/D these techniques facilitate separation and concentration of proteins by electrophoretic SCODA. Handle molecules may be attached to native proteins or protein fragments or to proteins that have been pretreated by one or more physical and/or chemical treatments as described above, for example.

### Experimental Example of Protein Focusing by Electrophoretic SCODA

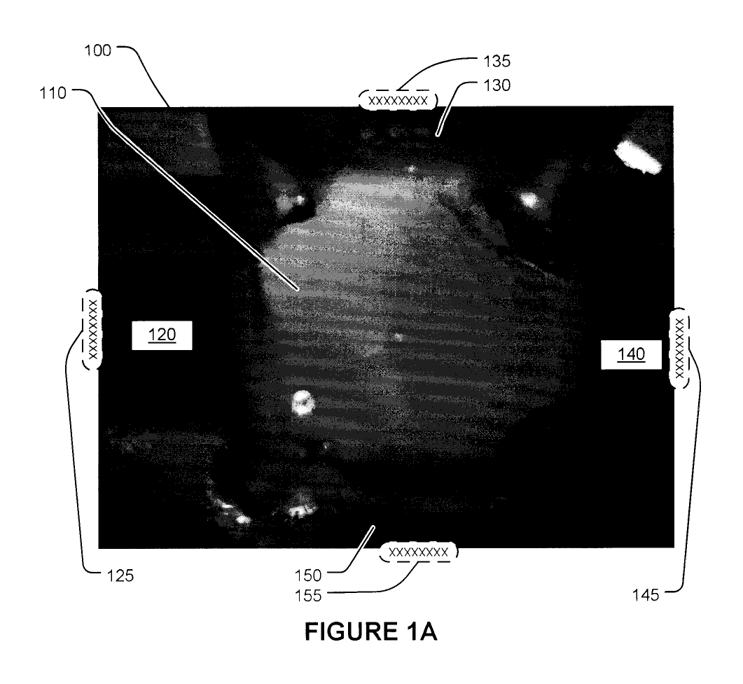

FIG. 1A illustrates an apparatus 100 used in an experiment where proteins were successfully concentrated by electrophoretic SCODA. Apparatus 100 comprises a polyacrylamide gel 110. Sample chambers 120, 130 are provided on either side of gel 110. Buffer reservoirs 140, 150 contain a buffer. Electrodes 125, 135, 145, 155 (indicated schematically and not to scale) are each immersed in a corresponding one of sample chambers 120, 130 and buffer reservoirs 140, 150. Electrodes 125, 135, 145, 155 were connected to different channels of a programmable power supply configured to apply sequences of electrical potentials to electrodes 125, 135, 145, 155 to provide electrical SCODA fields in gel 110.

A sample was prepared using pre-stained protein molecular weight marker commercially available from New England Biolabs Inc. (‘NEB’) of Ipswich, Mass., United States of America. The marker included proteins in several different molecular weight bands ranging from 6.5 to 175 kDa. The proteins in the marker were covalently coupled to a bromophenol blue dye which makes the protein bands visible in an electrophoretic gel under white light.

100 μL of NEB pre-stained Protein Marker, Broad Range (NEB #P7707s) was treated with 90 μL 5×Tris Glycine solution (125 mM Tris-Cl, 1.25M Glycine, 0.5% SDS), 10 μL 800 mM Dithiothreitol (‘DTT’) and 10 μL 10% SDS. The sample was heated by submersion in a boiling water bath for 3 minutes to denature the protein, and then added to 600 μL of distilled H₂0. It is thought that: the heating denatures proteins in the sample, SDS binds to the denatured proteins imparting negative charges to the protein molecules, and DTT prevents rebinding of disulphide bonds in the protein reducing the secondary structure of the protein. The sample was then loaded into sample chambers 120, 130 of SCODA apparatus 100.

In this experiment, gel 110, was a 3.5% polyacrylamide (29:1 crosslink ratio) gel prepared with 0.15×TBE buffer. Gel 110 was 16 mm wide and was prepared using a custom gel cap and side dams to limit oxygen exposure of the acrylamide and enhance polymerization.

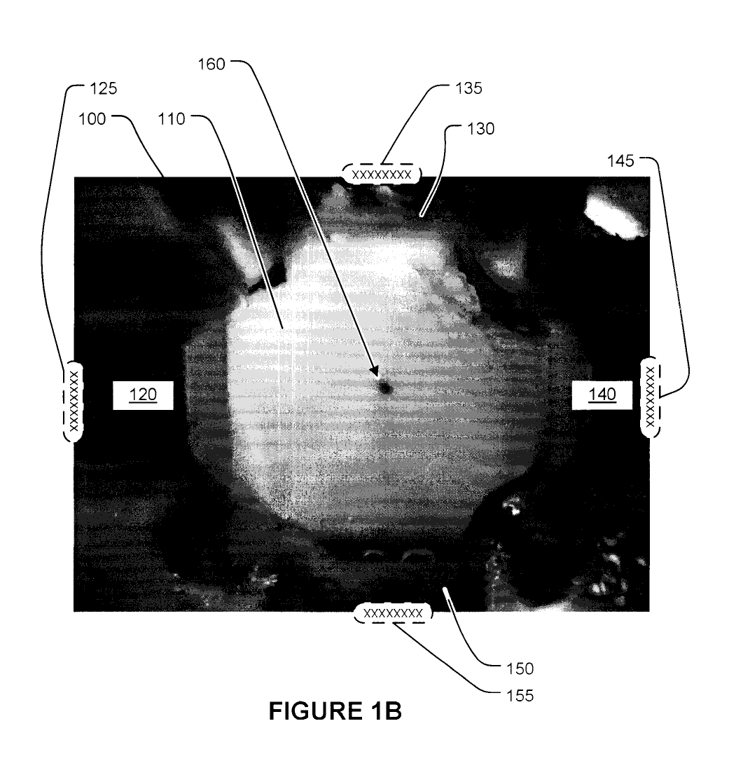

In this experiment the power supply was configured to apply voltages as set out in FIG. 2A. The values in FIG. 2A are voltages for power supply channels A, B, C, and D given in volts. Power supply channels A, B, C, and D are connected to electrodes 125, 135, 145, 155 such that channels A and C are connected to electrodes that are opposed to one another across gel 110 and channels B and D are connected to electrodes that are opposed to one another across gel 110. Any electrode of apparatus 100 may be connected to channel A.

In this experiment, the voltages at Time 1 were applied for 0.5 s, Time 2 for 1 s, Time 3 for 0.5 s, and Time 4 for 1 s, for a total run time of 1.5 hrs. The applied voltages drive protein molecules into gel 110 and concentrate protein molecules in gel 110. The voltages of FIG. 2A yield electric fields that provide a SCODA Dipole/Quadrupole ratio of 1.35, and a Quadrupole/Injection ratio of 5.

It was found that, under the influence of the SCODA fields, the pre-stained protein molecular weight markers from the samples loaded in sample chambers 120, 130, were focused in a region of gel 110. Gel 110 is free from protein in FIG. 1A. Following SCODA concentration as described above, gel 110 was imaged under white light. A stain indicating the presence of concentrated proteins was observed at a location 160 of gel 110, as shown in FIG. 1B.

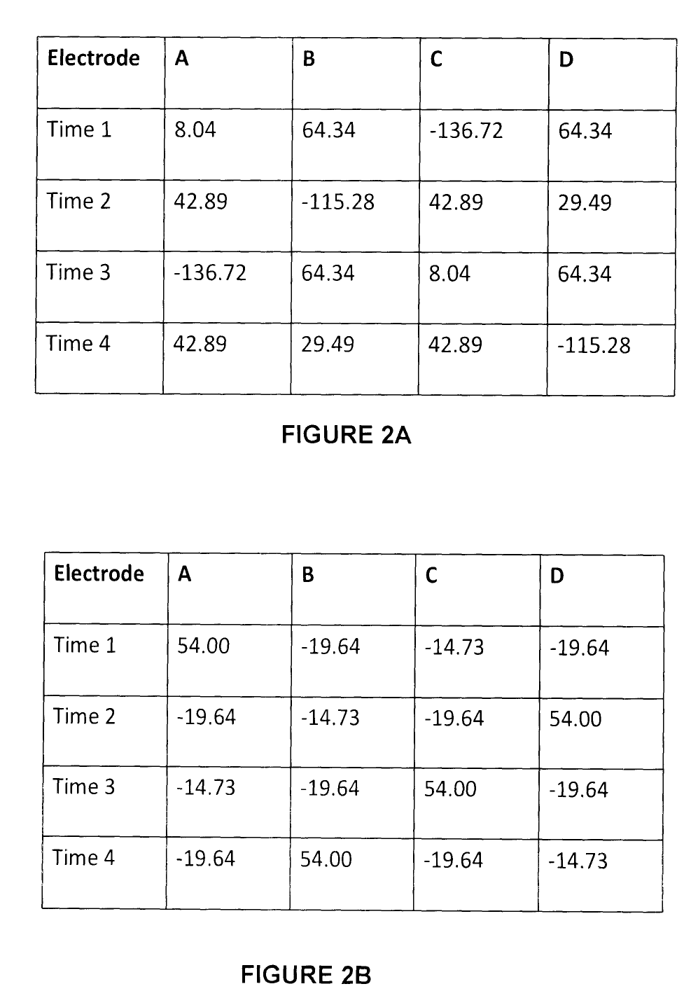

A portion of gel 110 including location 160 was cut out. A DC electrophoresis analysis of the extracted sample was performed using an SDS-PAGE gel. Control samples consisting of the ladders which comprised the original sample were also run on the SDS-PAGE gel. FIG. 3 shows the results of such work. The central lane shown in FIG. 3 contains the material extracted from location 160 of gel 110 (SCODA focus). This lane has one notable band. The left and right lanes shown in FIG. 3 contain the original sample. These lanes have bands corresponding to the proteins present in the original sample. It can be seen from FIG. 3 that the spot at location 160 of gel 110 contained 175 kDa and possibly 80 kDa proteins. Shorter proteins from the sample were not detected at spot 160 as indicated by the absence of bands in the center lane of FIG. 3 corresponding to such shorter proteins that are shown in the left and right lanes of FIG. 3.

This result demonstrates that electrophoretic SCODA may be applied to concentrate proteins and also that electrophoretic SCODA may be applied to separate longer proteins from shorter proteins.

Injection of Protein-Containing Samples into SCODA Media

In some embodiments, target particles are injected into a SCODA medium, such as a suitable gel, by techniques including electrokinetic injection, quadrupole injection and the like which use applied electrical fields to cause target particles to move from a sample into the SCODA medium. Such techniques can be inefficient or may not work at all in cases where the target particles are electrically neutral or have only small net electrical charges.

In one embodiment, a sample containing proteins which it is desired to concentrate is pre-treated as described above. The pre-treatment causes the target proteins to respond to the electrical fields applied to inject the target proteins into a SCODA medium. For example, injection and concentration of positively charged proteins from a sample may be achieved using combined electrokinetic injection, as discussed in PCT Publication No. 2006/081691 and suitable SCODA fields.

When apparatus like that in FIG. 1A is used, the proteins may be contained in samples in one or both of sample chambers 120 and 130. A DC voltage may be applied across gel 110 such that charged particles in either of sample chambers 120 and 130 flow into gel 110. Once the charged particles have entered gel 110, sequences of voltages may be applied to electrodes 125, 135, 145, 155 to perform SCODA concentration. FIG. 2B illustrates an example voltage sequence. Channels A through D may be connected to electrodes 125, 135, 145, 155 as described above.

With the FIG. 2B voltage sequence, the voltages at Time 1 may be applied for 1 s, Time 2 for 1 s, Time 3 for 1 s, and Time 4 for 1 s, for a total run time of 2 hrs. The fields correspond to a SCODA Dipole/Quadrupole ratio of 1.75. Electrokinetic injection may be completed prior to the application of SCODA fields to gel 110, or elecrokinetic injection and SCODA electric fields may be superimposed upon each other.

Focusing Two or More Types of Target Particle from a Sample

In some embodiments, two or more different groups of target particles from a single sample may be focused at different locations. In some embodiments the two groups of target particles are focused simultaneously. In other embodiments different groups of target particles are focused sequentially.

The different groups of target particles may have different charges. For example, particles of the first group of target particles may be positively charged while particles of a second group of target particles may be negatively charged.

The first group of target particles may comprise, in some embodiments, a protein which has been pre-treated to have a positive electrical charge. For example a sample may be pre-treated with cetyl trimethylammonium bromide (CTAB) such that a net positive charge is given to protein molecules in the sample. The second type of target particles may comprise, in some embodiments, a protein which has, or has been pre-treated to have, a negative electrical charge. For example, a sample containing proteins may be treated with SDS such that a net negative charge is given to protein molecules in the sample. Optionally, “handle” molecules are attached to one or both of the groups of target particles. The handle molecules may carry a net charge. For example, the handle molecules may comprise DNA or RNA. The DNA or RNA may have a net negative charge. In some embodiments, one or both of the first and second groups of target particles comprise nucleic acids such as DNA or RNA.

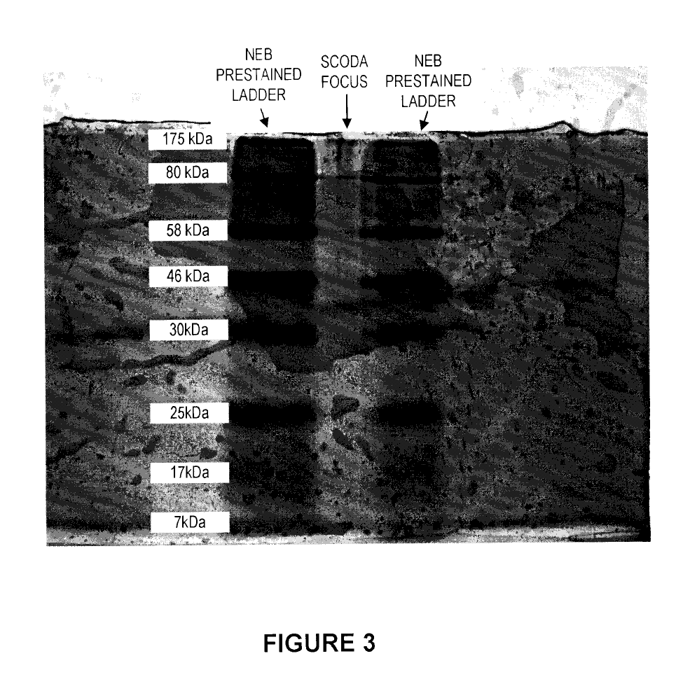

FIG. 4 shows apparatus 400 comprising first and second gels 410 and 420. A sample injection chamber 430 is located between first gel 410 and second gel 420. In the illustrated embodiment, buffer reservoirs 440, 450, 460, 470, 480, and 490 are provided around gels 410, 420. Electrodes 435, 445, 455, and 465 are in contact with a corresponding of one of sample chamber 430 and buffer reservoirs 440, 450, and 460. Electrodes 435, 445, 455, and 465 are connected to channels of a power supply that is configured or configurable to apply voltages to the electrodes that vary in time to provide electric SCODA fields in gel 410.

Additional electrodes 475, 485, 495 are in contact with buffer reservoirs 470, 480, and 490. SCODA electric fields may be provided in second gel 420 by applying suitable voltages to electrodes 435, 475, 485, and 495. This may be achieved by connecting electrodes 435, 475, 485 to suitable channels of a programmable power supply, for example. Under the influence of SCODA fields, positively charged target particles in the sample contained in chamber 430 may be injected into gel 410 and may be focused in a region of gel 410, and negatively charged particles from the sample may be simultaneously injected into gel 420 and focused in a region of gel 420.

In some embodiments, a DC bias voltage is applied across injection chamber 430 to drive positively charged particles in one direction while simultaneously driving negatively charged particles in the opposite direction. Such a DC bias voltage may be applied, for example, by making electrode 455 negative relative to electrode 485.

In a further embodiment, quadrupole injection may drive the first group of target particles from injection chamber 430 into gel 410 and quadrupole injection may drive the second group of target particles into gel 420.

As an example application, consider the case where a sample in sample chamber 430 contains both DNA and a protein. Such a sample may be obtained, for example by lysing a bacteria culture. In an illustrative example, a sample containing DNA and proteins could be created by lysing 0.5 mL of E. coli DH10B culture with CTAB-Bacterial Lysis Buffer (CTAB-BCB). CTAB-BCB may comprise 10 mM Tris HCl, 100 mM Na EDTA, 20 mM mercaptoethanol and 2% CTAB (Cetyl trimethylammonium bromide). In this illustrative example, treatment with CTAB may yield a sample solution containing positively-charged proteins and electrically-neutralized DNA. An amount, for example, 100 μL of the solution may be added to sample chamber 430 of apparatus 400. The electrically charged protein may then be injected into gel 410 by way of electrokinetic injection while the electrically neutral DNA remains in sample chamber 430. The protein may be concentrated at a location in gel 410 by scodaphoresis.

Subsequently, the DNA may be treated so that the DNA acquires an electrical charge. This treatment may be performed in sample chamber 430. For example, 50 μL of 20% SDS may be added to sample chamber 430. The DNA may become negatively charged as a result of interaction with the SDS. Electrokinetic injection may then be used to cause the DNA sample to enter gel 420. The DNA may be concentrated at a location in gel 420 by scodaphoresis.

Negative and positively charged particles may be injected with either quadrupole injection fields and electrokinetic injection fields. The above embodiments are merely exemplary. Other quadrupole injection/electrokinetic injection and SCODA fields may be used to inject and concentrate sample outputs within apparatus 400. Further, apparatus 400 may have additional or fewer sample chambers and buffer reservoirs. In some embodiments, additional electrodes may be present in apparatus 400. In some embodiments, three or more gels or other SCODA media are arranged around a common sample chamber.

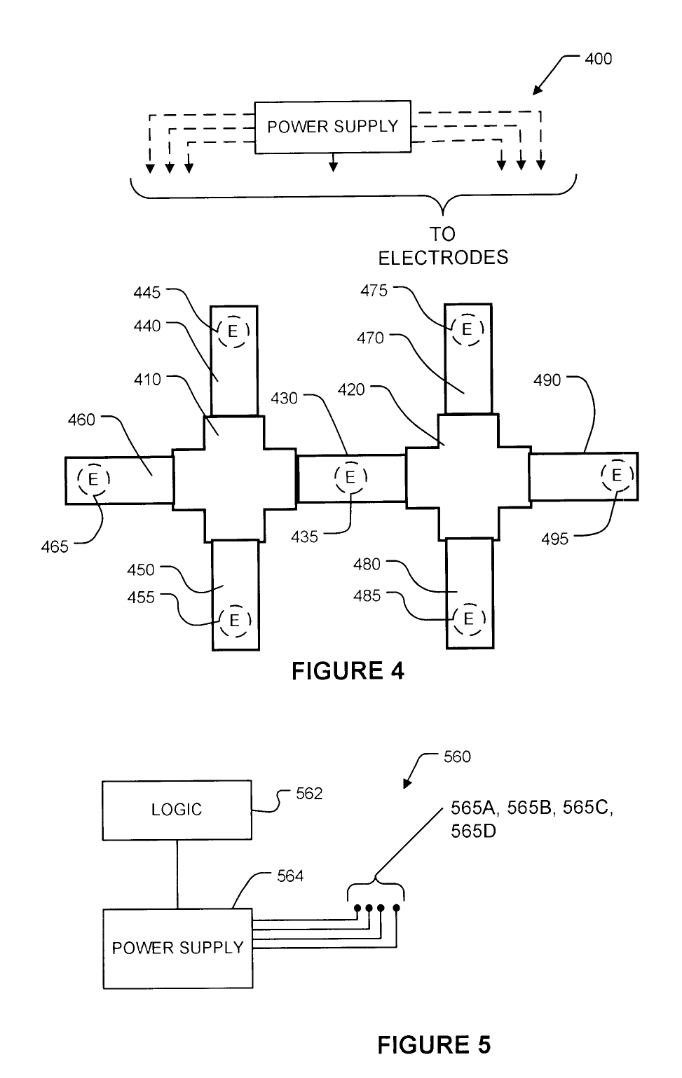

FIG. 5 shows a SCODA controller 560 according to another example embodiment. Controller 560 comprises a logic unit 562 which may comprise a data processor executing software instructions, hard-wired logic circuits, a suitably-configured configurable logic device (such as a field-programmable gate array), a suitable combination thereof, or the like. Logic unit 562 controls the operation of a power supply 564 having multiple outputs 565. In the illustrated embodiment, power supply 464 has four independently-controllable outputs 565A to 565D. Other embodiments may provide a different number of outputs. In yet other embodiments one output of power supply 564 is not independently controllable. Outputs 565 may be connected to electrodes such as electrodes 125, 135, 145, 155, associated with a SCODA matrix such as gel 110.

### Sample Conductivity-Reduction

There are cases in which it is desirable to use SCODA to concentrate target particles from samples that have relatively high electrical conductivity. For example:
A sample may be pre-treated in a high salinity buffer for lysis and for inactivation of nucleases.
It may be desirable to prepare samples using chaotropic agents such as guanidinium hydrochloride and guanidinium thiocyanate or other chaotropic agents.
It may be desirable to prepare a sample using one or more highly conductive anionic surfactants (detergents) such as SDS (sodium dodecyl sulphate).
   The resulting samples may have a very high electrical conductivity. It is difficult to inject target particles from a highly conductive sample by techniques which apply electrical fields for particle injection because the high conductivity of the sample reduces the electrical fields within the sample. Increasing the potential difference across the sample to increase the electrical fields inside the sample results in electrical current flow through the sample which cases heating and can damage target particles in some cases. Using smaller electrical fields results in long injection times and poor SCODA performance. It is generally desirable to match the electrical conductivity of the sample to that of the SCODA medium (e.g. gel) being used.

As a non-limiting example, the conductivity of a SCODA medium may be chosen to have an electrical conductivity that is high enough that electric fields in one or more sample chambers adjacent to the SCODA medium are sufficient to inject target particles into the SCODA medium in a desirably short time. If the electrical conductivity of the SCODA medium is very low then most electrical potential may be dropped across the SCODA medium resulting in undesirably small electric injection fields and undesirably long injection times. On the other hand, the electrical conductivity of the SCODA medium is desirably sufficiently low that applied SCODA electric fields do not cause too much heating of the SCODA medium. In some embodiments the SCODA medium comprises a gel having an electrical conductivity of 250 μS/cm. In some embodiments, the SCODA medium has an electrical conductivity of a few hundred μS/cm or lower (e.g. 200 to 800 μS/cm or less). In some embodiments the SCODA medium has an electrical conductivity of 300 μS/cm or less. For fast efficient injection of target particles into the SCODA medium it is desirable that the sample have an electrical conductivity that is smaller than that of the SCODA medium.

Embodiments provide SCODA methods which include a step for reducing electrical conductivity of a sample containing target particles. In some embodiments, the conductivity-reducing step comprises a buffer exchange step performed prior to injection of target particles into a SCODA medium. The buffer exchange step transfers the target particles from a sample having higher electrical conductivity to a sample having a lower electrical conductivity such as a low salinity buffer. In some embodiments the buffer exchange step is also effective to lyse cells in the sample and preferentially extract nucleic acids or other biomolecules of interest. In some embodiments the conductivity-reducing step comprises a step which removes or neutralizes charge carriers (such as salts) from a sample which also contains target particles. Removal or neutralization of the charge carriers reduces electrical conductivity of the sample. In some embodiments the conductivity-reducing step reduces electrical conductivity of the sample to a level that is on the order of or less than the electrical conductivity of the SCODA medium.

### Example Embodiments Using Buffer Exchange

In some embodiments a buffer exchange step comprises placing a sample containing target particles in contact with a solid material to which target particles bind, separating the solid material from the remaining sample fluid, placing the solid material and associated target particles in contact with a low-conductivity buffer, and transferring the target particles into the low-conductivity buffer.

For example, where the target particles comprise DNA, the buffer exchange step may comprise incubation of a high salinity sample containing DNA in the presence of a DNA-binding matrix such as diatomaceous earth or silica gel under chemical conditions that cause nucleic acids to bind to the DNA-binding matrix. Once binding has occurred, the high salinity sample is separated from the DNA-binding matrix and a low salinity buffer is added. The DNA is caused to unbind from the DNA-binding matrix by providing suitable conditions in the low-salinity buffer. For example, under high pH conditions DNA unbinds from diatomaceous earth. The low salinity buffer, with or without the DNA-binding matrix, is placed in a SCODA sample chamber. SCODA DNA extraction and concentration is then performed.

In some embodiments, DNA is recovered from the DNA-binding matrix by washing the matrix to remove contaminants, as well as any remaining high-conductivity material from the sample. After washing, elution of the DNA with a suitable low ionic strength elution buffer or water, under suitable conditions (e.g. neutral or slightly basic pH), may be completed. The elution buffer may be ideally suited for SCODA and therefore the solid-phase step acts as an ideal buffer-exchange step in the SCODA system, converting the sample from high salinity buffer to low-salinity buffer.

The combination of SCODA with solid-phase DNA extraction can provide advantages over other DNA separation and concentration methods. Because SCODA can extract target particles from relatively very dilute samples, a relatively large amount of matrix can be used to bind DNA or other target particles. This is not practical where DNA separation is performed by other methods which cannot handle large elution volumes. Additionally, SCODA may reject certain contaminants that carry through the solid-phase purification.

Standard purification methods using DNA-binding matrices, such as silica gel, are limited to using small amounts of matrix because the matrix volume determines the final elution volume, which must be small to avoid excessively low concentration of the eluted product. In addition, elution occurs through fluid flow which limits elution time and elution efficiency. Also, contaminants often carry through such methods due to indiscriminate binding to the matrix.

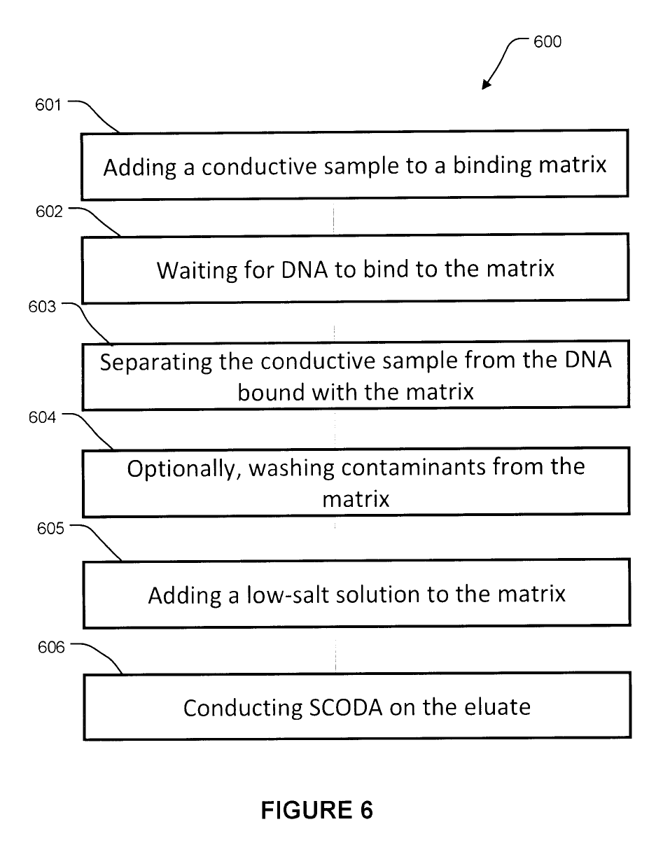

FIG. 6 is a flow diagram of a method 600 for concentrating target particles from within a high conductivity mixture. Method 600 comprises: binding DNA to a matrix under high salt lysis conditions; followed by washing to remove the high salt buffers; followed by elution of the binding matrix with low salt buffer; followed by SCODA concentration of the target particles from the low salt buffer. As the sample will be concentrated and further purified by SCODA, the matrix may be eluted in a much larger volume of low salt buffer than is practical with other separation techniques. This improves the extraction efficiency of the solid-phase step. In some embodiments the binding matrix us placed directly into a sample chamber of a SCODA apparatus. In such embodiments, any DNA or other target particles still weakly bound to the binding matrix will experience the electric fields being applied to inject target particles into the SCODA medium, possibly further improving the efficiency of DNA recovery.

At block 601, a highly conductive sample, such as a lysate containing 4M guanididium, is added to an amount of DNA-binding matrix (such as diatomaceous earth or silica gel). In some embodiments, a large amount of DNA-binding matrix may be used, such as several millilitres or several hundred microlitres. This is not mandatory, however. In other embodiments, smaller amounts of DNA-binding matrix may be used.

At block 602, DNA present in the highly-conductive sample is allowed to bind to the DNA-binding matrix.

At block 603, the DNA-binding matrix is separated from the highly-conductive sample. In some embodiments block 603 comprises centrifugally separating the matrix from the sample. For example, the sample and matrix may be introduced into a spin column from which fluid may be spun out by a centrifugal system. In further embodiments, fluid may be separated from the matrix by filtering, by means of a gravity flow system, positive pressure system, or a vacuum manifold.

Block 604 provides an optional washing step. For example, block 604 may comprise washing the separated matrix with an ethanol containing buffer. Such a buffer may wash salt and other contaminants from the matrix.

At 605, a low salt elution buffer is brought into contact with the matrix to elute the DNA from the matrix. In some embodiments, block 605 comprises using a relatively large amount of low salt elution buffer (for example several millilitres or several hundred microlitres). In an example embodiment, the elution buffer has a volume on the order of 5 mL. In some embodiments, the volume of the elution buffer is substantially the same as a capacity of a SCODA sample chamber. For example, the SCODA sample chamber may have a capacity of a few mL (e.g. 5 mL) and in block 605 the target particles may be eluted into a few mL (e.g. 5 mL) of a low-salt buffer. In other embodiments smaller amounts of low salt elution buffer may be used.

At 606, the eluate is purified and concentrated by SCODA. The SCODA can proceed more quickly and efficiently and with less heating than would be possible if one attempted to perform SCODA by placing the highly conductive unprocessed starting sample in a sample chamber of SCODA apparatus like that described above (for example sample chamber 120 and/or 130 shown in FIG. 1). Target particles may be injected from the eluate into a SCODA medium, for example, by a quadrupole injection field or a electrokinetic injection field.

### Buffer Exchange Working Examples

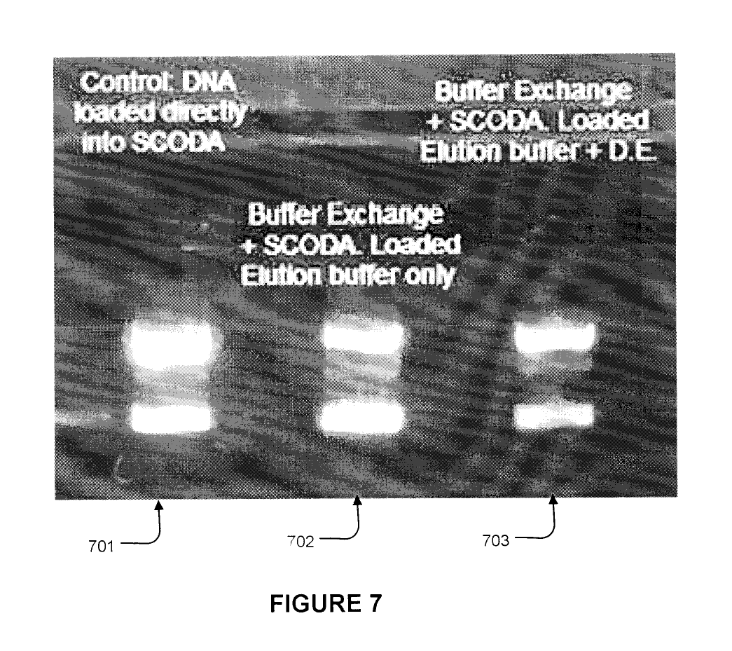

FIG. 7 shows an image of a DC electrophoresis gel 700 showing DNA recovered from a sample by a SCODA method that included performing a buffer exchange. The leftmost column 701 is a control lane showing the input DNA, the center column 702 is the DNA extracted by SCODA from the eluate resulting from incubation of the sample with diatomaceous earth and subsequent elution. The rightmost column 703 is DNA extracted using SCODA from a mixture of the diatomaceous earth and elution buffer.

To prepare gel 700, 100 μL of 5 ng/μL pUC19 DNA in TE buffer was mixed with 400 μL of distilled H₂0, 575 μL of 8M Guanidine HCl and 75 μL of 200 mg/mL diatomaceous earth in a microcentrifuge tube. This mixture was vortexed vigorously for 30 s and spun down on a microcentrifuge for 30 s. The supernatant was removed, being careful not to disturb the binding matrix, and 1000 μL of 95% ethanol was added to the tube. The mixture was then vortexed for 30 s and spun down for 30 s, and the supernatant carefully removed. The final elution involved addition of 1 mL TE buffer, vortexing for 30 s, spinning down for 30 s and removal of the supernatant, repeated twice. The resulting buffer was then diluted to 5 mL with distilled H₂0 and loaded directly into a sample chamber of SCODA apparatus. In the run resulting in column 703 the diatomaceous earth was introduced into the sample chamber. In the run resulting in column 702 the diatomaceous earth was removed before the diluted supernatant was introduced into the SCODA sample chamber.

The SCODA medium in each case was a 1% low melting point agarose SCODA gel in 0.25×TBE. The DNA was injected into the SCODA medium using an electric field of 20V/cm for 20 minutes. The DNA was then concentrated to a tightly focused spot using 50V/cm SCODA fields having a 4s rotational period for 2 hrs. The focused DNA was extracted from the SCODA gel and run in a DC gel against a control 701 to confirm DNA recovery, shown in FIG. 1A. The control 701 was the same input sample to the buffer exchange, 100 μL of 5 ng/μL pUC19 DNA, but run only through SCODA. Large variation in fluorescence quantification in gels make it difficult to determine exact recovery efficiencies, but the gel indicates high efficiency from loading only the elution buffer into SCODA, as well as from loading the elution buffer and diatomaceous earth.

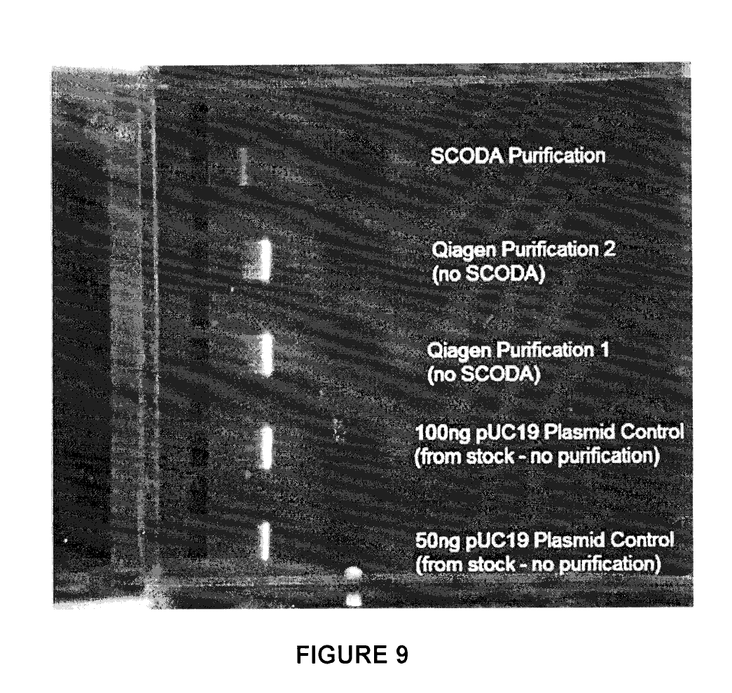

FIG. 9 shows a DC electrophoresis gel showing DNA recovered by SCODA from lysed E. coli. The lysis was performed in a high salinity buffer. DNA was recovered using a buffer exchange process generally like that described as method 600.

To extract plasmid DNA from E. coli, 5 mL of overnight culture was spun down to form a pellet of cells, and supernatant removed. 400 μL of 1×TE was added to the resulting pellet, and 575 μL of 8M Guanidine HCl and 75 μL of 200 mg/mL diatomaceous earth were added as well. The mixture was then vortexed vigorously for 30 s after which it was allowed to sit at room temperature for 1 hr to allow lysis to proceed. After 1 hr the mixture was vortexed again for 30 s, spun down and supernatant removed. The DNA was then eluted in 1 mL of 1×TE, vortexed for 30 s and spun down. 1 mL of the solution containing the eluted DNA was loaded into a sample chamber adjacent to a SCODA gel. The sample was eluted again with 1 mL 1×TE, vortexed, and the resulting mixture, including diatomaceous earth, loaded directly into SCODA with the previous 1 mL sample. The SCODA gel comprised a 1% low melting point agarose SCODA gel in 0.25×TBE.

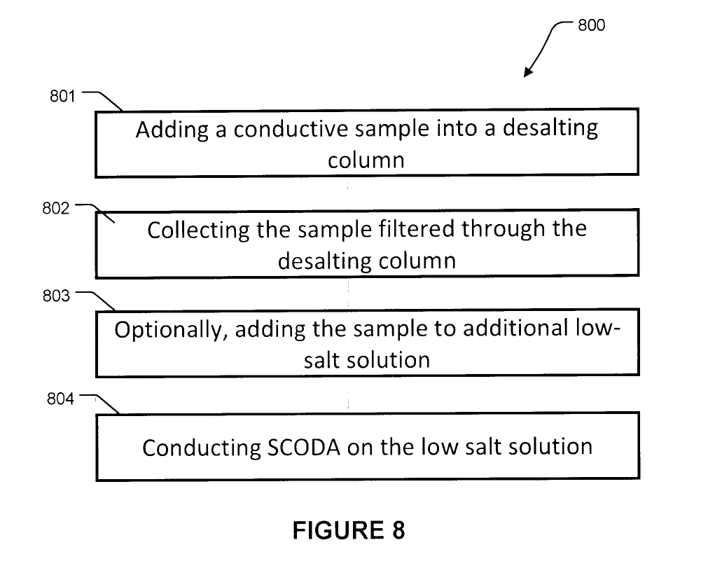

The DNA was then injected into the SCODA gel with a 20V/cm electric field applied for 20 minutes. The DNA was concentrated using 60V/cm SCODA fields with 4 s rotational periods for 1.5 hrs to yield a tightly focused spot. The focused DNA was extracted from the SCODA gel and run in a DC gel against a control to confirm DNA recovery as shown in FIG. 8. Different amounts of each sample were loaded, so fluorescence cannot be directly compared. Results were compared against 2 Qiagen Miniprep™ extraction kits designed for plasmid extraction from E. coli. Fluorescent analysis shows that the SCODA process is about 50% as efficient as the Qiagen kits in this situation. It should be noted, however that SCODA samples generally fluoresce less on a DC gel after having been previously concentrated with SYBR green. With this buffer exchange step, SCODA purification time from E. coli is reduced from 4 hours to 1.5 hours because of the reduction in sample buffer salinity.

### Example Embodiments Applying Desalination to Reduce Conductivity

FIG. 8 is a flow diagram of a method 800 for concentrating target particles such as a DNA, RNA, or proteins from within a high conductivity sample. Method 800 comprises treating a high conductivity mixture containing target particles to remove chemical species that cause the high electrical conductivity. In an example embodiment, method 800 comprises passing the high conductivity sample through a desalting column, such as a Sephadex™ desalting column, available from General Electric Healthcare of Piscataway, N.J., United States of America. A low conductivity fraction containing the target particles can then be subjected to SCODA.

At block 801, a highly conductive solution containing target particles (for example a lysate containing 4M guanididium) is passed into a desalting column. The desalting column may contain a low salinity solution into which the highly conductive sample is added.

At block 802, the sample is filtered through the desalting column. Longer molecules, such as DNA, RNA, and other target particles of interest may pass through the desalting column more quickly than salt particles. By collecting a fraction of the fluid emerging from the column which is known to contain the target particles and not collecting the fraction of the fluid which follows, the target particles are separated into a low-conductivity fluid. Block 802 may, for example, comprise taking a predetermined volume of fluid that has flows from the desalting column. As the target particles will be concentrated and further purified by SCODA, the fraction taken from the desalting column may contain contaminants which would make the fraction unusable for other concentration protocols.

At optional block 803, the runoff from the desalting column is diluted further with a volume of low salinity solution. In some embodiments, a relatively large amount of low salinity solution may be used, such as several millilitres or several hundred microlitres. In other embodiments a smaller amount, or no, low salinity solution is added.

In some embodiments, the desalting column may discharge the fraction containing target particles directly into a volume of low salinity solution or into a sample of a SCODA apparatus (e.g. sample chamber 120 or 130 of FIGS. 1A and 1B). In some embodiments, discharge from the desalting column is automatically shut off after one or more of:
a predetermined amount of time has elapsed since the sample was introduced into the desalting column;
a predetermined volume of fluid has exited the desalting column;
electrical conductivity of the fluid exiting the desalting column has started to increase and/or has reached or exceeds a threshold conductivity.
   In some embodiments an automatically controlled valve blocks flow from the desalting column to prevent further discharge of the column from entering the sample injection chamber.

At block 804, the low salinity solution is purified and concentrated using SCODA. As in method 600, SCODA injection and concentration can proceed more quickly and efficiently than would be the case if one attempted to inject the target particles into a SCODA medium directly from the initial highly-conductive unprocessed sample. Target particles may be injected into a SCODA medium, for example, by a quadrupole injection field or a electrokinetic injection field.

Methods 600 and 800 may be automated by providing suitable controllers which operate valves and fluid transfer devices to process samples as described above and to deliver the processed samples into a chamber from which target particles can be injected into a SCODA medium.

Embodiments which apply methods like method 600 and/or method 800 can have significant advantages over prior methods for separating DNA or other target particles from samples. These advantages may include:
Simplified lysis procedures may be used to release target particles. SCODA can reject many contaminants that would interfere with other separation technologies.
Relatively large elution volumes (for example, a few mL) may be used since SCODA can concentrate target particles from very dilute samples.
In embodiments where target particles are bound to a binding matrix during buffer exchange, relatively large volumes (surface area) of binding matrix may be used as compared to conventional solid phase extraction. SCODA can tolerate large elution volumes. For example, in some embodiments, elution volumes can be on the order of 5 mL. Larger binding matrix surface areas may allow for larger quantities of DNA or other target particles to be processed in a single run.
In some embodiments the binding matrix may be placed directly into a SCODA sample chamber. This may simplify the buffer exchange process by eliminating the need to separate the elution supernatant and the binding matrix. This may also increase the efficiency with which target particles are recovered.
In some embodiments that apply a buffer exchange process, binding matrix is introduced directly to an elution buffer without a wash step. This simplifies buffer exchange. Contaminants that would have been removed by a wash step may be rejected by SCODA.
   It is not mandatory that any of these advantages be present in any particular embodiment.

Some embodiments combine features of two or more of the embodiments described above. For example, in some embodiments, the target particles are protein molecules and the embodiments concentrate the protein molecules by methods that comprise:
Performing one or more chemical and/or physical pre-treatment steps that result in the protein molecules being more strongly affected by electric fields and/or enhance the non-linearity of the response of the protein molecules to applied electric fields;
optionally, add handle molecules to the protein molecules;
performing a step for reducing electrical conductivity of the resulting sample (this step may comprise buffer exchange and/or separation of electrically conductive contaminant species for example); and
concentrating the target protein molecules from the resulting sample by SCODA.
   Similar embodiments may also be applied to the concentration of target particles other than protein molecules.

In other example embodiments the target particles are protein molecules and the embodiments concentrate the protein molecules by methods that comprise:
Adding handle molecules to the protein molecules. The Handle molecules optionally comprise nucleic acids;
performing a step for reducing electrical conductivity of the resulting sample (this step may comprise buffer exchange and/or separation of electrically conductive contaminant species for example). In some embodiments the step for reducing electrical conductivity comprises a buffer exchange step using a binding matrix that has a particular affinity for the handle molecules; and
concentrating the target protein molecules from the resulting sample by SCODA.
   Similar embodiments may also be applied to the concentration of target particles other than protein molecules.

In embodiments like any of those described herein, one or more reagents such as heparin may be added to the sample prior to performing SCODA to concentrate target particles from the sample. Such reagents may help to improve DNA yield from some samples. For example, adding heparin to a sample may help to saturate binding sites on molecules that could otherwise bind to DNA and prevent DNA from injecting into the SCODA gel. Since SCODA may be able to preferentially concentrate DNA and reject heparin from the concentrated product, the addition of heparin to the sample may improve DNA injection without carrying through to the final product.

While some of the embodiments described above have particular advantages in cases where the target particles are protein molecules or fragments of protein molecules or nucleic acids such as DNA or RNA, the methods and apparatus described herein may be applied to target particles of other types that can be concentrated by SCODA, for example, nanoparticles, polystyrene particles, polysaccharides, lipids, vitamins, hormones, carbohydrates and the like.

The foregoing detailed description has set forth various embodiments of the devices and/or processes via the use of block diagrams, schematic illustrations, flowcharts and examples. Insofar as such block diagrams, schematic illustrations, flowcharts and examples contain one or more functions and/or operations, it will be understood by those skilled in the art that each function and/or operation within such block diagrams, schematic illustrations, flowcharts or examples can be implemented, individually and/or collectively. Methods, or processes set out herein, may include acts performed in a different order, may include additional acts and/or omit some acts.

Features of the various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification, including but not limited to U.S. Patent Publication No. 2009/0139867, PCT Publication No. 2006/081691 and PCT Publication No. WO 2009/094772 are incorporated herein by reference, in their entirety. Aspects of the example embodiments disclosed herein may be modified to employ features of systems, circuits and concepts disclosed in the incorporated patents, applications and publications to provide yet further example embodiments.

Any of the techniques described may optionally be applied to concentrate target particles in wells within SCODA media as described, for example, in PCT Publication No. WO 2009/094772. In such embodiments, target particles may enter a fluid in a well and may be withdrawn by extracting fluid from the well.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

While a number of exemplary aspects and embodiments have been discussed above, those of skill in the art will recognize certain modifications, permutations, additions and sub-combinations thereof. It is intended that the following appended claims and claims hereafter introduced are interpreted to include all such modifications, permutations, additions and sub-combinations as are within their true spirit and scope.

1. A method for concentrating a selected protein from a sample, the method comprising:
treating the sample, the treating increasing a response of the selected protein to an applied electric field;
applying one or more electric fields to inject the selected protein from the sample into a scodaphoresis medium; and
applying electric scodaphoresis fields to the scodaphoresis medium to concentrate the selected protein at a location in the scodaphoresis medium, the scodaphoresis fields comprising a time-varying driving field that applies forces to the selected protein and a time-varying mobility-altering field that affects a mobility of the selected protein.

2. A method according to claim 1 wherein treating the sample comprises denaturing the selected protein.

3. A method according to claim 1 wherein the treatment comprises chemically treating the sample with one or more of: tris glycine; dithiothreitol, sodium dodecyl sulfate, and cetyl trimethylammonium bromide.

4. A method according to claim 1 wherein the treatment comprises heating the sample.

5. A method according to claim 1 wherein the treatment increases a net electrical charge on molecules of the selected protein.

6. A method according to claim 5 wherein, after the treatment, molecules of the selected protein have a positive net electrical charge.

7. A method according to claim 5 wherein, after the treatment, molecules of the selected protein have a negative net electrical charge.

8. A method according to claim 1 further comprising linking at least one handle molecule to the selected protein in the sample prior to injecting the selected protein from the sample into the gel.

9. A method according to claim 8 wherein the handle molecule comprises a linking agent selected from the group consisting of:
an antibody,
an antigen,
a biotin-avidin complex, and
an RNA aptamer.

10. A method according to claim 8 wherein linking the handle molecule to the selected protein comprises establishing between the handle molecule and the selected protein one or more of:
at least one hydrogen bond;
at least one ionic bond;
at least one hydrophobic interaction; and,
at least one covalent bond.

11. A method according to claim 8 wherein the handle molecule comprises one or both of DNA and RNA.

12. A method for concentrating target particles, the method comprising:
providing a sample containing the target particles wherein the sample has an electrical conductivity exceeding an electrical conductivity of a scodaphoresis medium;
performing a conductivity reducing step on the sample to produce a reduced-conductivity sample containing the target particles and having an electrical conductivity equal to or less than the conductivity of the scodaphoresis medium;
applying one or more electric fields to inject the target particles from the reduced-conductivity sample into the scodaphoresis medium; and
applying scodaphoresis fields to the scodaphoresis medium to concentrate the target particles at a location in the scodaphoresis medium.

13. A method according to claim 12 wherein the conductivity-reducing step comprises
injecting the sample into a desalting column; and
collecting the reduced conductivity sample at an outlet of the desalting column.

14. A method according to claim 13 comprising automatically collecting discharge from the desalting column until one or more of:
a predetermined amount of time has elapsed since the sample was introduced into the desalting column;
a predetermined volume of fluid has exited the desalting column; and
electrical conductivity of fluid exiting the desalting column has started to increase and/or has reached or exceeds a threshold conductivity.

15. A method according to claim 12 wherein performing the conductivity reducing step comprises:
contacting the high conductivity sample with a binding matrix and allowing the target particles to bind to the binding matrix;
separating the binding matrix from the sample; and,
eluting the target particles to yield the reduced-conductivity sample.

16. A method according to claim 15, further comprising, after separating the binding matrix and prior to eluting the target particles:
rinsing the binding matrix under conditions wherein the target particles remain bound to the binding matrix during the rinsing.

17. A method according to claim 15 comprising placing the binding matrix and reduced-conductivity sample together in a sample chamber adjacent to the scodaphoresis medium.

18. A method according to claim 15 wherein the binding matrix comprises at least one of: diatomaceous earth and silica gel.

19. A method according to claim 15 further comprising:
diluting the reduced-conductivity sample with a low salt buffer prior to or during injecting the target particles from the reduced-conductivity sample into the scodaphoresis medium.

20. A method according to claim 12 wherein the reduced-conductivity sample has a volume of at least 100 microlitres.

21. A method according to claim 12 wherein the sample comprises a reagent and one or both of injecting the target particles from the reduced-conductivity sample into a scodaphoresis medium; and concentrating the target particles at a location in the scodaphoresis medium comprises rejecting the reagent.

22. A method according to claim 21 wherein the reagent comprises heparin.

23. A method according to claim 12 wherein the target particles comprise at least one of DNA and RNA, and the sample contains sodium dodecyl sulphate.

24. A method according to claim 12 wherein the scodaphoresis medium has an electrical conductivity of 300 μS/cm or less.

25. Apparatus for concentrating first and second types of target particle from a sample, the apparatus comprising:
a sample chamber for receiving the sample;
first and second bodies of a scodaphoresis medium adjacent to the sample chamber;
a scodaphoresis controller connected to apply at least one of a first scodaphoresis field to the first body of scodaphoresis medium and a second scodaphoresis field to the second body of scodaphoresis medium.

26. Apparatus according to claim 25 wherein the scodaphoresis controller is configured to apply an electrical injection field to the sample chamber.

27. Apparatus according to claim 26 wherein target particles of the first type of target particles have a positive net charge, target particles of the second type of target particles have a negative net charge, and the electrical injection field is operative to inject the target particles of the first type of target particles into the first body of scodaphoresis medium and to inject the target particles of the second type of target particles into the second body of scodaphoresis medium.

28. Apparatus according to claim 25 wherein the scodaphoresis controller comprises a power supply connected to supply electrical potentials to a plurality of electrodes spaced apart around a circumference of at least one of the first and second bodies of scodaphoresis medium.

29. A method for concentrating first and second types of target particle from a sample, the method comprising:
placing the sample in a sample chamber located between first and second bodies of a scodaphoresis medium;
applying one or more injection fields to the sample chamber, the injection fields operative to drive target particles of the first type into the first body of scodaphoresis medium and to drive target particles of the second type into the second body of scodaphoresis medium;
concentrating the target particles of the first type by applying scodaphoresis fields to the first body of scodaphoresis medium and concentrating the target particles of the second type by applying scodaphoresis fields to the second body of scodaphoresis medium.

30. A method according to claim 29 wherein the target particles of the first and second types have opposite electrical charges and the injection field comprises an electrical injection field.

31. A method according to claim 30 comprising simultaneously driving the target particles of the first type into the first body of scodaphoresis medium and driving the target particles of the second type into the second body of scodaphoresis medium.

32. A method according to claim 30 comprising:
driving the target particles of the first type into the first body of scodaphoresis medium;
treating the target particles of the second type to make the target particles of the second type responsive to the one or more injection fields; and,
driving the treated target particles of the second type into the second body of scodaphoresis medium.

33. A method according to claim 29 comprising treating the sample to cause target particles of at least one of the first and second types to have a net electrical charge.

34. A method according to claim 33 wherein treating the sample comprises imparting a net positive electrical charge to the target particles of at least one of the first and second types.

35. A method according to claim 33 wherein treating the sample comprises imparting a net negative electrical charge to the target particles of at least one of the first and second types.

36. A method according to claim 33 wherein treating the sample comprises imparting a net negative electrical charge to the target particles of one of the first and second types and imparting a positive electrical charge to the target particles of the other one of the first and second types.

37. A method according to claim 29 wherein at least one of the first and second types of target particles comprises a protein.

38. A method according to claim 37 comprising denaturing the protein prior to concentrating the protein.

39. A method according to claim 37 comprising chemically treating the protein with least one of cetyl trimethylammonium bromide and sodium dodecyl sulfate prior to concentrating the protein.

40. A method according to claim 37 comprising binding at least one handle molecule to the protein prior to concentrating the protein.

41. A method according to claim 40 comprising binding the handle molecule to the protein by at least one of:
an antibody,
an antigen,
a biotin-avidin complex, and
an RNA aptamer.

42. A method according to claim 38 comprising binding the handle molecule to the protein by at least one of:
at least one hydrogen bond,
at least one ionic bond,
at least one hydrophobic interaction, and
at least one covalent bond
between the at least one handle molecule and the protein.

43. A method according to claim 39 wherein the at least one handle molecule comprises a nucleic acid.

44. A method according to claim 29 wherein at least one of the first and second types of target particles comprises DNA or RNA.

45. A method according to claim 29 wherein the sample comprises a reagent and any of:
driving target particles of the first type into the first body of scodaphoresis medium;
driving target particles of the second type into the second body of scodaphoresis medium;
concentrating the target particles of the first type; and
concentrating the target particles of the second type;
comprise rejecting the reagent.

46. A method according to claim 29 wherein the reagent comprises heparin.

47-48. (canceled)
